(19) United States
(12) Patent Application Publication
(10) Pub. No.: US 2023/0120923 A1
(43) Pub. Date: Apr. 20, 2023

(54) Breast Pump Belt Assembly

(71) Applicant: Mary Meyers, Woodbury, MN (US)

(72) Inventor: Mary Meyers, Woodbury, MN (US)

(21) Appl. No.: 17/504,963

(22) Filed: Oct. 19, 2021

(51) Int. Cl. A61M 1/06; A45F 3/14

(52) U.S. Cl. CPC A61M 1/067; A45F 3/14; A45F 2003/144

(57) ABSTRACT

A breast pump belt assembly for securing a breast pump to a user’s waist includes a first belt which has a prominence integrated into the first belt. A buckle is coupled to the first belt. A second belt is coupled to and is oriented coextensive with the first belt. The second belt is extendable through the buckle to secure the first belt around the user’s waist. A pair of couplers is each movably integrated into the first belt. A strap is provided which extends through each of the couplers on the first belt. The strap is matable to itself such that the strap can be extended through a handle of a breast pump thereby facilitating the breast pump to be attached to the first belt.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### Field of the Invention

The disclosure relates to belt devices and more particularly pertains to a new belt device for securing a breast pump to a user’s waist. The device includes a belt that is wearable around a user’s waist and a strap that is integrated into the belt. The strap engages a pair of couplers on the belt such that each of a pair of ends of the strap are directed toward each other. The strap is matable to itself thereby facilitating the strap to be extended through a handle of a breast pump. In this way the breast pump can be mounted to the belt thereby facilitating the breast pump to be portable.

### Description of Related Art Including Information Disclosed Under 37 CFR 1.97 and 1.98.

The prior art relates to belt devices including a brassiere that includes nipple caps and a reservoir that is in fluid communication with the nipple caps for collecting breast milk in a discrete manner. The prior art discloses a belt which includes a slots for accommodating respective cones of a breast pump for mounting the breast pump to a user. The prior art discloses a breast pump device that can be integrated into an existing brassiere. The prior art discloses a neck strap for supporting a breast pump.

### BRIEF SUMMARY OF THE INVENTION

An embodiment of the disclosure meets the needs presented above by generally comprising a first belt which has a prominence integrated into the first belt. A buckle is coupled to the first belt. A second belt is coupled to and is oriented coextensive with the first belt. The second belt is extendable through the buckle to secure the first belt around the user’s waist. A pair of couplers is each movably integrated into the first belt. A strap is provided which extends through each of the couplers on the first belt. The strap is matable to itself such that the strap can be extended through a handle of a breast pump thereby facilitating the breast pump to be attached to the first belt.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S

The disclosure will be better understood and objects other than those set forth above will become apparent when consideration is given to the following detailed description thereof. Such description makes reference to the annexed drawings wherein:

FIG. 1 is a front perspective view of a breast pump belt assembly according to an embodiment of the disclosure.

FIG. 2 is a back view of an embodiment of the disclosure.

FIG. 3 is a front view of an embodiment of the disclosure.

FIG. 4 is a top perspective view of an embodiment of the disclosure.

FIG. 5 is a perspective view of an embodiment of the disclosure showing a strap being extended through a handle of a breast pump.

FIG. 6 is a perspective in-use view of an embodiment of the disclosure.

### DETAILED DESCRIPTION OF THE INVENTION

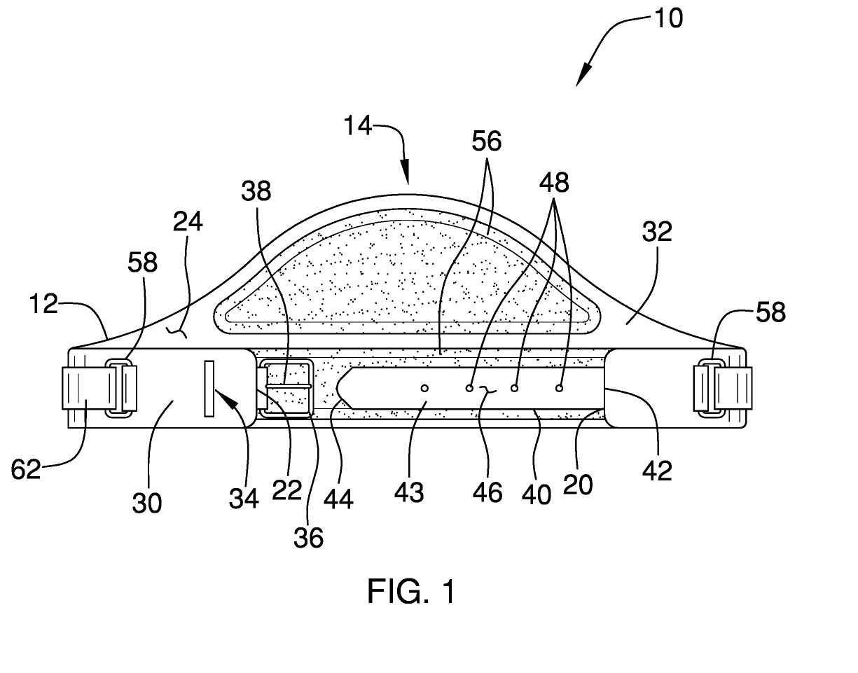

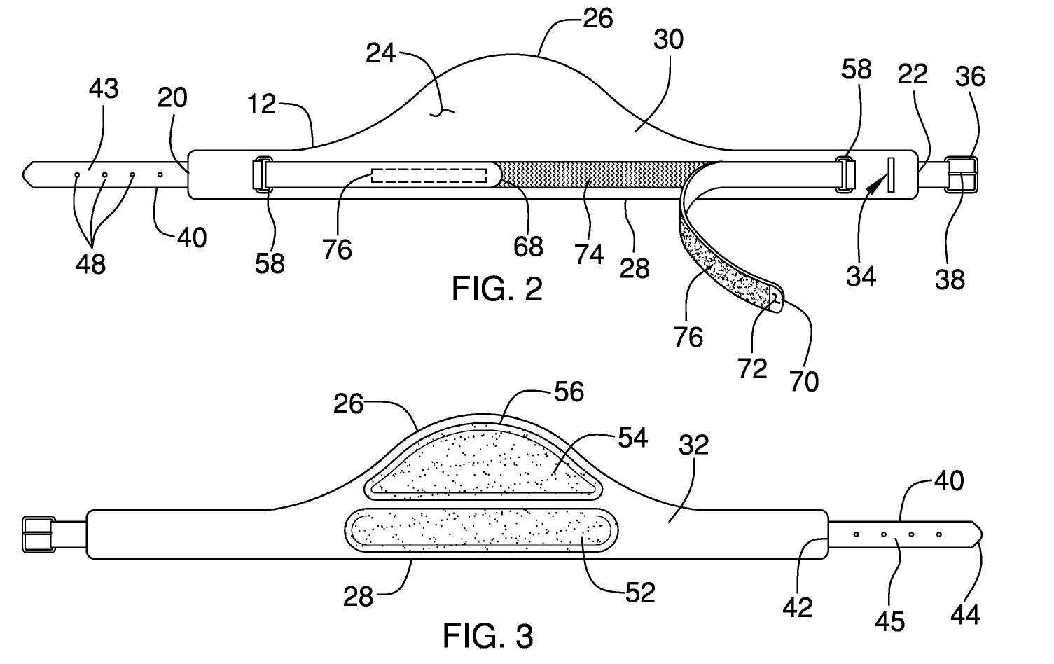

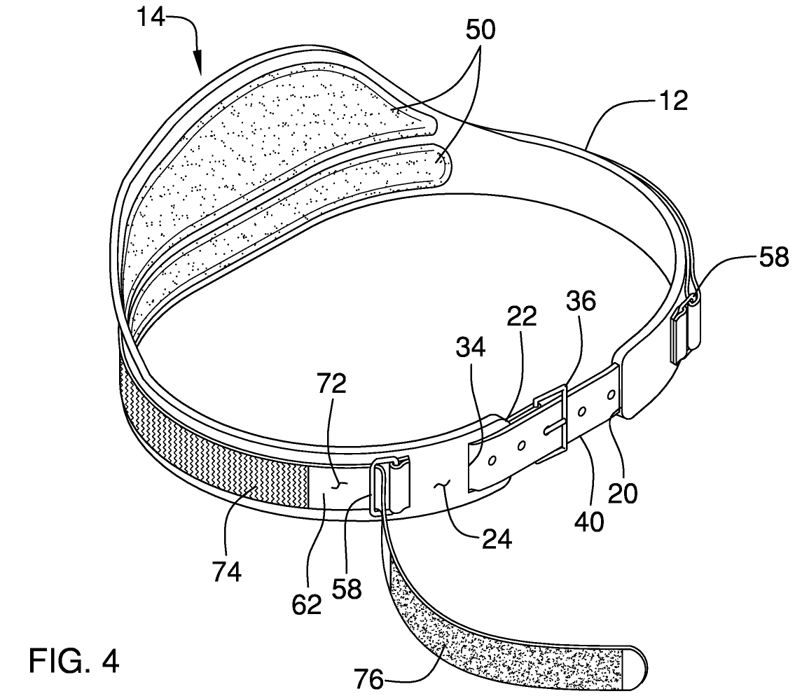

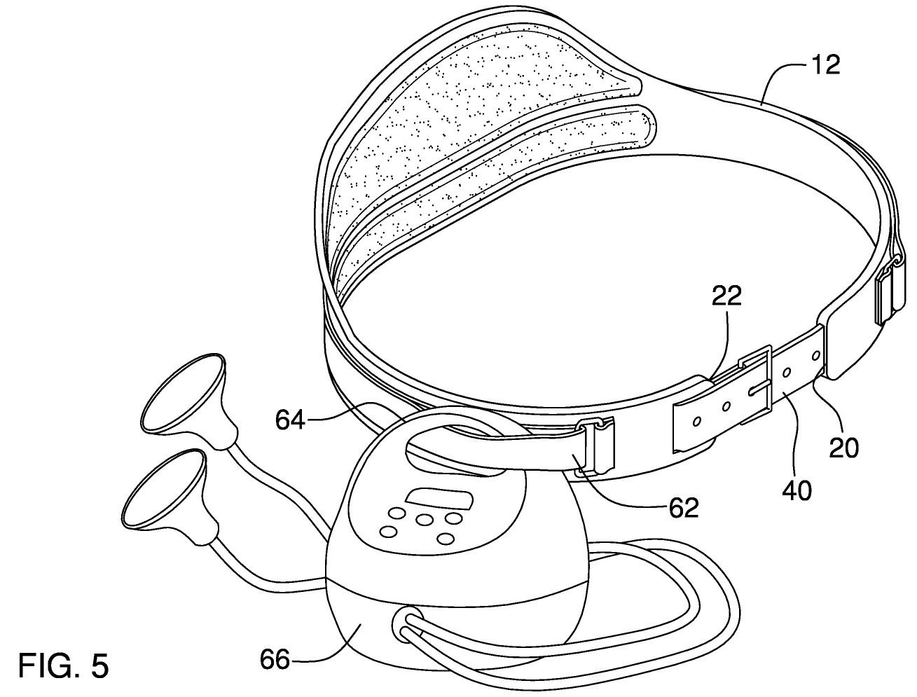

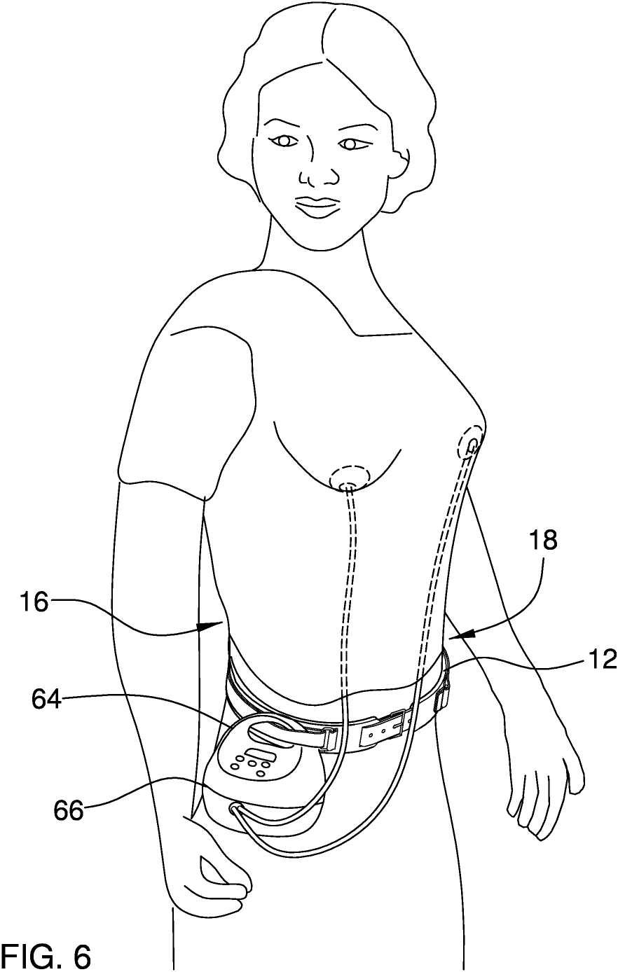

With reference now to the drawings, and in particular to FIGS. 1 through 6 thereof, a new belt device embodying the principles and concepts of an embodiment of the disclosure and generally designated by the reference numeral 10 will be described.

As best illustrated in FIGS. 1 through 6, the breast pump belt assembly 10 generally comprises a first belt 12 that has a prominence 14 which is integrated into the first belt 12. The prominence 14 extends along a user’s back 16 when the first belt 12 is worn around the user’s waist 18. The user may be a female that is breast feeding an infant and who regularly employs a breast pump. The first belt 12 has a first end 20, a second end 22 and an outer surface 24 extending between the first end 20 and the second end 22. The first belt 12 has a top edge 26 extending between the first end 20 and the second end 22 and the first belt 12 has a bottom edge 28 extending between the first end 20 and the second end 22. Furthermore, the top edge 26 curves upwardly and downwardly on opposite sides of a vertical axis that is centrally positioned between the first end 20 and the second end 22 to define the prominence 14 on the first belt 12.

The outer surface 24 has a front side 30 and a back side 32, and the front side 30 has a slot 34 extending into an interior of the first belt 12. The slot 34 extends substantially between the top edge 26 and the bottom edge 28 of the first belt 12. Additionally, the slot 34 is positioned adjacent to the first end 20 of the first belt 12. A buckle 36 is coupled to the first belt 12 and the buckle 36 is movably disposed on the first end 20 of the first belt 12. Furthermore, the buckle 36 includes a finger 38 is movably disposed on the buckle 36. The buckle 36 may comprise a belt buckle that is commonly employed on waist belts that are worn on pants.

A second belt 40 is coupled to and is oriented coextensive with the first belt 12. The second belt 40 is extendable through the buckle 36 to secure the first belt 12 around the user’s waist 18. The second belt 40 has a coupled end 42, a free end 44 and an outside surface 46 extending between the coupled end 42 and the free end 44. The outside surface 46 has a front side 43 and a back side 45, and the second belt 40 has a plurality of holes 48 each extending through the front side 43 and the back side 45 of the outside surface 46 of the second belt 40. The holes 48 are spaced apart from each other and are distributed between the coupled end 42 and the free end 44. The coupled end 42 is coupled to the first end 20 of the first belt 12 and the free end 44 is insertable through the buckle 36. The finger 38 on the buckle 36 is extendable through a respective one of the holes 48 thereby facilitating the buckle 36 to adjust a diameter of a closed loop formed by the first belt 12 and the second belt 40. In this way the buckle 36 facilitates the first belt 12 to accommodate a variety of sizes of waists. The free end 44 of the second belt 40 is insertable into the slot 34 in the first belt 12 when the second belt 40 is extended through the buckle 36 for storing the free end 44.

A pair of pads 50 is provided and each of the pads 50 is coupled to the first belt 12. Each of the pads 50 is aligned with the prominence 14 such that each of the pads 50 abuts the user’s back 16 when the first belt 12 is worn. Each of the pads 50 is comprised of a resiliently compressible material to enhance comfort for the user. Each of the pads 50 is positioned on the back side 32 of the outer surface 24 of the first belt 12. The pair of pads 50 includes a first pad 52 that is positioned adjacent to the bottom edge 28 of the first belt 12. The pair of pads 50 includes a second pad 54 that is positioned between the first pad 52 and the top edge 26 of the first belt 12. Additionally, the second pad 54 has an upper edge 56 that is co-arcuate with the top edge 26 of the first belt 12 defining the prominence 14.

A pair of couplers 58 is each movably integrated into the first belt 12 and each of the couplers 58 is disposed on the front side 30 of the outer surface 24 of the first belt 12. Each of the couplers 58 is spaced from a respective one of the first end 20 and the second end 22 of the first belt 12. Each of the couplers 58 is continuous such that each of the couplers 58 forms a closed loop. Each of the couplers 58 extends through a respective one of a pair of engagement points 60 that is each integrated into the front side 30 of the outer surface 24 of the first belt 12.

A strap 62 is provided and the strap 62 extends through each of the couplers 58 on the first belt 12. The strap 62 is matable to itself thereby facilitating the strap 62 to be extended through a handle 64 of a breast pump 66 thereby facilitating the breast pump 66 to be attached to the first belt 12. The strap 62 has a primary end 68, a secondary end 70 and an exterior surface 72 extending between the primary end 68 and the secondary end 70. Each of the primary end 68 and the secondary end 70 is extended through a respective one of the couplers 58 such that the primary end 68 and the secondary end 70 are directed toward each other on the first belt 12.

A first mating member 74 is coupled to the exterior surface 72 of the strap 62. The first mating member 74 is centrally positioned on the strap 62 such that the first mating member 74 extends substantially between each of the couplers 58 on the first belt 12. A pair of second mating members 76 is provided and each of the second mating members 76 is coupled to the exterior surface 72 of the strap 62. Each of the second mating members 76 extends between the first mating member 74 and a respective one of the primary end 68 and the secondary end 70 of the strap 62. Moreover, each of the second mating members 76 is releasably matable to the first mating member 74. A respective one of the primary end 68 or the secondary end 70 of the strap 62 is extendable through the handle 64 of the breast pump 66 such that the breast pump 66 is attached to the first belt 12 when the second mating members 76 are attached to the first mating member 74. Additionally, each of the first mating member 74 and the second mating member may comprise complementary portions of a hook and loop fastener or the like.

In use, the first belt 12 is wrapped around the user’s waist 18, the second belt 40 is engaged to the buckle 36 and the second belt 40 is inserted into the slot 34 in the first belt 12. Either the primary end 68 of the strap 62 or the secondary end 70 of the strap 62 is extended through the handle 64 of the breast pump 66, depending on the user’s preference. Moreover, the strap 62 is mated to itself when the strap 62 is inserted through the handle 64 of the breast pump 66 to mount the breast pump 66 to the first belt 12. In this way the breast pump 66 is portable thereby facilitating the user to pump breast milk while the user is working or performing any other activity outside of their home. Furthermore, the breast pump 66 can be positioned at a variety of locations on the first belt 12 to accommodate the user’s preferences.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A breast pump belt assembly for supporting a breast pump on a user’s waist thereby facilitating the breast pump to be portable, said assembly comprising:
a first belt having a prominence being integrated into said first belt wherein said prominence is configured to extend along a user’s back when said first belt is worn around the user’s waist;
a buckle being coupled to said first belt;
a second belt being coupled to and being oriented coextensive with said first belt, said second belt being extendable through said buckle wherein said second belt is configured to secure said first belt around the user’s waist;
a pair of couplers, each of said couplers being movably integrated into said first belt; and
a strap extending through each of said couplers on said first belt, said strap being matable to itself wherein said strap is configured to be extended through a handle of a breast pump thereby facilitating the breast pump to be attached to said first belt.

2. The assembly according to claim 1, wherein:
said first belt has a first end, a second end and an outer surface extending between said first end and said second end, said first belt having a top edge extending between said first end and said second end, said first belt having a bottom edge extending between said first end and said second end, said top edge curving upwardly and downwardly on opposite sides of a vertical axis being centrally positioned between said first end and said second end to define said prominence on said first belt, said outer surface having a front side and a back side; and
said front side has a slot extending into an interior of said first belt, said slot extending substantially between said top edge and said bottom edge of said first belt, said slot being positioned adjacent to said first end of said first belt.

3. The assembly according to claim 2, wherein:
said buckle is movably disposed on said first end of said first belt, said buckle including a finger being movably disposed on said buckle; and
said second belt has a coupled end, a free end and an outside surface extending between said coupled end and said free end, said outside surface having a front side and a back side, said second belt having a plurality of holes each extending through said front side and said back side, said holes being spaced apart from each other and being distributed between said coupled end and said free end;
said coupled end being coupled to said first end of said belt, said free end being insertable through said buckle having said finger on said buckle extending through a respective one of said holes thereby facilitating said buckle to adjust a diameter of a closed loop formed by said first belt and said second belt wherein said buckle is configured to accommodate a variety of sizes of waists; and
said free end of said second belt is insertable into said slot in said first belt when said second belt is extended through said buckle for storing said free end.

4. The assembly according to claim 2, further comprising a pair of pads, each of said pads being coupled to said first belt, each of said pads being aligned with said prominence wherein each of said pads is configured to abut the user’s lower back when said belt is worn, each of said pads being comprised of a resiliently compressible material wherein said pair of pads is configured to enhance comfort for the user.

5. The assembly according to claim 4, wherein each of said pads is positioned on said back side of said outer surface of said first belt, said pair of pads including a first pad being positioned adjacent to said bottom edge of said first belt, said pair of pads including a second pad being positioned between said first pad and said top edge of said first belt, said second pad having an upper edge being co-arcuate with said top edge of said first belt defining said prominence.

6. The assembly according to claim 2, wherein each of said couplers is disposed on said front side of said outer surface of said first belt, each of said couplers being spaced from a respective one of said first end and said second end of said first belt, each of said couplers being continuous such that each of said couplers forms a closed loop, each of said couplers extending through a respective one of a pair of engagement points each being integrated into said front side of said outer surface of said first belt.

7. The assembly according to claim 1, wherein said strap has a primary end, a secondary end and an exterior surface extending between said primary end and said secondary end, each of said primary end and said secondary end being extended through a respective one of said couplers such that said primary end and said secondary end are directed toward each other on said first belt.

8. The assembly according to claim 7, further comprising a first mating member being coupled to said exterior surface of said strap, said first mating member being centrally positioned on said strap such that said first mating member extends substantially between each of said couplers on said first belt.

9. The assembly according to claim 8, further comprising a pair of second mating members, each of said second mating members being coupled to said exterior surface of said strap, each of said second mating members extending between said first mating member and a respective one of said primary end and said secondary end of said strap.

10. The assembly according to claim 9, wherein each of said second mating members is releasably matable to said first mating member, a respective one of said primary end or said secondary end of said strap being extendable through the handle of the breast pump such that the breast pump is attached to said first belt when said second mating members are attached to said first mating member.

11. A breast pump belt assembly for supporting a breast pump on a user’s waist thereby facilitating the breast pump to be portable, said assembly comprising:
a first belt having a prominence being integrated into said first belt wherein said prominence is configured to extend along a user’s back when said first belt is worn around the user’s waist, said first belt having a first end, a second end and an outer surface extending between said first end and said second end, said first belt having a top edge extending between said first end and said second end, said first belt having a bottom edge extending between said first end and said second end, said top edge curving upwardly and downwardly on opposite sides of a vertical axis being centrally positioned between said first end and said second end to define said prominence on said first belt, said outer surface having a front side and a back side, said front side having a slot extending into an interior of said first belt, said slot extending substantially between said top edge and said bottom edge of said first belt, said slot being positioned adjacent to said first end of said first belt;
a buckle being coupled to said first belt, said buckle being movably disposed on said first end of said first belt, said buckle including a finger being movably disposed on said buckle;
a second belt being coupled to and being oriented coextensive with said first belt, said second belt being extendable through said buckle wherein said second belt is configured to secure said first belt around the user’s waist, said second belt having a coupled end, a free end and an outside surface extending between said coupled end and said free end, said outside surface having a front side and a back side, said second belt having a plurality of holes each extending through said front side and said back side, said holes being spaced apart from each other and being distributed between said coupled end and said free end, said coupled end being coupled to said first end of said belt, said free end being insertable through said buckle having said finger on said buckle extending through a respective one of said holes thereby facilitating said buckle to adjust a diameter of a closed loop formed by said first belt and said second belt wherein said buckle is configured to accommodate a variety of sizes of waists, said free end of said second belt being insertable into said slot in said first belt when said second belt is extended through said buckle for storing said free end;
a pair of pads, each of said pads being coupled to said first belt, each of said pads being aligned with said prominence wherein each of said pads is configured to abut the user’s lower back when said belt is worn, each of said pads being comprised of a resiliently compressible material wherein said pair of pads is configured to enhance comfort for the user, each of said pads being positioned on said back side of said outer surface of said first belt, said pair of pads including a first pad being positioned adjacent to said bottom edge of said first belt, said pair of pads including a second pad being positioned between said first pad and said top edge of said first belt, said second pad having an upper edge being co-arcuate with said top edge of said first belt defining said prominence;
a pair of couplers, each of said couplers being movably integrated into said first belt, each of said couplers being disposed on said front side of said outer surface of said first belt, each of said couplers being spaced from a respective one of said first end and said second end of said first belt, each of said couplers being continuous such that each of said couplers forms a closed loop, each of said couplers extending through a respective one of a pair of engagement points each being integrated into said front side of said outer surface of said first belt;
a strap extending through each of said couplers on said first belt, said strap being matable to itself wherein said strap is configured to be extended through a handle of a breast pump thereby facilitating the breast pump to be attached to said first belt, said strap having a primary end, a secondary end and an exterior surface extending between said primary end and said secondary end, each of said primary end and said secondary end being extended through a respective one of said couplers such that said primary end and said secondary end are directed toward each other on said first belt;
a first mating member being coupled to said exterior surface of said strap, said first mating member being centrally positioned on said strap such that said first mating member extends substantially between each of said couplers on said first belt; and
a pair of second mating members, each of said second mating members being coupled to said exterior surface of said strap, each of said second mating members extending between said first mating member and a respective one of said primary end and said secondary end of said strap, each of said second mating members being releasably matable to said first mating member, a respective one of said primary end or said secondary end of said strap being extendable through the handle of the breast pump such that the breast pump is attached to said first belt when said second mating members are attached to said first mating member.
